(19) United States
(12) Patent Application Publication
(10) Pub. No.: US 2023/0338463 A1
(43) Pub. Date: Oct. 26, 2023

(54) ICD PEPTIDE AND USE THEREOF FOR HAVING CYTOTOXIC ACTIVITY ON CANCER CELLS

(71) Applicant: NATIONAL TSING HUA UNIVERSITY, HSINCHU (TW)

(72) Inventors: JYA-WEI CHENG, HSINCHU (TW); CHIH-LUNG WU, HSINCHU (TW); YU-HUAN CHEAH, HSINCHU (TW); Chun-Yu LIU, HSINCHU (TW)

(21) Appl. No.: 18/336,518

(22) Filed: Jun. 16, 2023

Related U.S. Application Data
(63) Continuation-in-part of application No. 17/727,276, filed on Apr. 22, 2022.

(51) Int. Cl. A61K 38/10; A61P 35/00

(52) U.S. Cl. CPC A61K 38/10; A61P 35/00

(57) ABSTRACT

The present invention provides a pharmaceutical composition and method for having cytotoxic activity to interact with cancer cells and destroy the cancer cells.

### CROSS-REFERENCE TO RELATED APPLICATION

This application is a continuation-in-part of the U.S. Application Ser. No. 17/727,276, filed Apr. 22, 2022, which is herein incorporated by reference in its entirety.

### REFERENCE TO AN ELECTRONIC SEQUENCE LISTING

The contents of the electronic sequence listing (THPA-2021001-US-CIP (ST26SequenceListing).xml; Size: 12.4 KB; and Date of Creation: 2023-06-13) is herein incorporated by reference in its entirety.

### FIELD OF THE INVENTION

This disclosure relates to peptide-based therapeutic compositions and methods for resulting in an anti-tumor response and the effective regression of the cancer.

### BACKGROUND OF THE INVENTION

Cancer imposes a global health burden as it represents one of the leading causes of morbidity and mortality while also giving rise to significant economic burden owing to the associated expenditures for its monitoring and treatment.

Cancer cells are characterized by uncontrolled proliferation and the ability to invade surrounding normal tissue or distant sites by homological and/or lymphatic spread. The difficulty for the effective treatment of cancer relates to establishing the distinction between malignant and normal cells of the body. Both are derived from the same source and are very similar, and for this reason, there is no significant recognition by the immune system as to the threat.

Despite the advances made in cancer therapy over the past few decades, such as surgery, radiotherapy, chemotherapy and immunotherapy, these therapeutic modalities are still associated with significant side effects.

It is an unmet need for anti-cancer treatments with less side effects and/or for chemotherapy resistant or immunotherapy resistant cancers. Therefore, the present invention addresses several inherent weaknesses that are in need of attention in the design and development of peptide-based cancer therapeutics owing to its favorable and intrinsic properties of being potent, safe and low in production costs.

### SUMMARY OF THE INVENTION

Accordingly, the present invention provides the search for new ways to treat cancer is a process in constant progress, and within this dynamic, new active principles were developed to be useful in the treatment of cancer.

Another aspect of the present invention provides methods for inhibiting or reducing cancer cells by administering to a subject in need thereof a therapeutically effective amount of the peptides.

In summary, these and other objects have been achieved according to the present disclosure which demonstrates the pharmaceutical composition attenuatimg cancer cells growth, inhibit cancer cells viability, lead to HMGB1 passive release from the cancer cells, lead to release of ATP from the cancer cells, lead to release of cytochrome c from the cancer cells, or/ and lead to release of ROS from the cancer cells.

Detailed description of the invention is given in the following embodiments with reference to the accompanying drawings.

### BRIEF DESCRIPTION OF THE DRAWINGS

The drawings described herein are for illustration purposes only and are not intended to limit the scope of the present teachings in any way.

FIGS. 1A-1F show that anticancer activities of SEQ ID NO:1, SEQ ID NO:2, SEQ ID NO:3, SEQ ID NO:4 and SEQ ID NO: 5 were administrated to cancer cells/ cancer cell lines via MTT cytotoxicity assay.

FIG. 1A represents the cytotoxicity activity of human non-small cell lung cancer cell PC9.

FIG. 1B represents the cytotoxicity activity of human non-small cell lung cancer cell H1975.

FIG. 1C represents the cytotoxicity activity of adenocarcinomic alveolar basal epithelial cells A549.

FIG. 1D represents the cytotoxicity activity of oral squamous cell carcinoma CGNHC9.

FIG. 1E represents the cytotoxicity activity of gingival epidermoid carcinoma OECM-1.

FIG. 1F represents anticancer activities of Nal-P-113, Bip-P-113 and Dip-P-113 against various cancer MN-11 cells by MTT cell viability assay. Data represent mean ± SD of three independent experiments.

FIG. 2 shows that SEQ ID NO: 1 peptide and its derivatives (SEQ ID NO:2, SEQ ID NO:3, SEQ ID NO:4 and SEQ ID NO: 5) were administrated to human fibroblast cells (HFW) via MTT cytotoxicity assay.

FIG. 3A shows that Time killing analysis of PC9 cells via administrated SEQ ID NO:3, SEQ ID NO:4 and SEQ ID NO: 5 peptides.

FIG. 3B shows that time killing analysis of Nal-P-113, Bip-P-113 and Dip-P-113 against MN-11 cell line. Data represent mean ± SD of three independent experiments.

FIGS. 4A-4C show that the HMGB 1 protein secretion from cell lysate to supernatant after the incubation with SEQ ID NO:3, SEQ ID NO:4 and SEQ ID NO: 5 after 1 hour (FIG. 4A), 2 hour (FIG. 4B) and 4 hour (FIG. 4C). “S” represented supernatant in FIGS. 4A-4C. “L” represented lysate in FIGS. 4A-4C.

FIGS. 4D-4E show that HMGB 1 protein secreted from cell lysate to supernatant after being treated with Nal-P-113, Bip-P-113 and Dip-P-113 at different time points. Untreated cells were used as a control.

FIG. 5A shows that ROS release after the treatment with 2 × IC₅₀ of SEQ ID NO:3, SEQ ID NO:4 and SEQ ID NO: 5 using DCFDA cellular ROS detection kit. Data were presented as means ± SD (standard deviation) and analyzed by the one-way ANOVA and Student’s t-test.

FIGS. 5B-5D shows that ROS release from MN-11 cells after the treatment with 2× IC₅₀ of Nal-P-113 (FIG. 5B), Bip-P-113 (FIG. 5C) and Dip-P-113 (FIG. 5D) detected by DCFDA cellular ROS detection kit. The untreated cells were used as control. Results are presented as mean ± SD of three independent experiments.

FIG. 6A shows that the ATP secretion of PC 9 administrated with SEQ ID NO:3, SEQ ID NO:4 and SEQ ID NO: 5 points was measured for 0.5, 1, 1.5 and 2 hours. Data are presented as means ± SD (standard deviation). Data of FIG. 6A were analyzed using the one-way ANOVA and Student’s t-test. Data were presented as means ± SD (standard deviation) and analyzed by the one-way ANOVA and Student’s t-test.

FIGS. 6B-6D shows that ATP release of MN-11 cells after treatment with 2× IC₅₀ Nal-P-113 (FIG. 6B), Bip-P-113 (FIG. 6C) and Dip-P-113 (FIG. 6D). Untreated cells were served as the control group. Results are presented as mean ± SD of three independent experiments, **P<0.01; ***P<0.001 compared with control.

FIGS. 7A-7C shows that the cytochrome c secretion from cancer cells administrated with SEQ ID NO:3 (FIG. 7A), SEQ ID NO:4 (FIG. 7B) and SEQ ID NO: 5 (FIG. 7C) was quantified using ELISA method.

FIGS. 8A-8C shows that the cytochrome c secretion was quantified using ELISA assay. The cells were treated with 2× IC₅₀ Nal-P-113 (FIG. 8A), Bip-P-113 (FIG. 8B), and Dip-P-113 (FIG. 8C) for 0.5, 1, 1.5, 2, and 4 h, respectively. Untreated cells were used as a control. Results are presented as mean ± SD of three independent experiments. **P<0.01; ***P<0.001 compared with control.

FIGS. 9A-9E shows that effects of Nal-P-113 on fibrosarcoma and its toxicity in C57BL/6 mice. Animal survival rate (FIG. 9A). Tumor volumes treated with sterile 0.9% NaCl (negative control) (FIG. 9B), 1 mg Nal-P-113 (FIG. 9C). Body weight was recorded from the first day of treatment (FIG. 9D). Intake was recorded from the first day of treatment (FIG. 9E). The survival curves were analyzed using a log-rank test. ***P<0.001 compared with negative control.

FIGS. 10A-10B shows that survival rate (FIG. 10A) and tumor growth (FIG. 10B) of cured animals re-challenged by MN-11 cells. Survival curves were analyzed using a log-rank test. **P<0.01 compared with negative control.

### DETAILED DESCRIPTION OF THE INVENTION

The following description is merely exemplary in nature and is in no way intended to limit the present teachings, application, of uses.

Optionally, in an exemplary embodiment of the present invention, the ICD peptides of the present invention include, but are not limited to, Sequences of SEQ ID NO: 1 - SEQ ID NO: 5.

The present invention provides a method for treating, preventing, or alleviating a symptom of cancer by administering to the subject a pharmaceutical composition, optionally in a therapeutically effective amount.

“Subjects” as used herein are generally human subjects and includes, but is not limited to, cancer patients. The subjects may be male or female and may be of any race or ethnicity. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g. mouse, rat, guinea pig, and hamster), lagomorphs, primates (including non-human primates), etc.

The cancers to which the cancer cell inhibitory pharmaceutical compositions of the present invention are applicable are not particularly limited. Examples of the cancers include lung cancer, oral cancer, fibrosarcoma, salivary gland cancer, tongue cancer, pharyngeal cancer, esophagus cancer, stomach cancer, pancreatic cancer, liver cancer, bile duct cancer, duodenal cancer, small intestine cancer, large bowel cancer, colon cancer, rectal cancer, renal cancer, bladder cancer, prostatic cancer, penile cancer, testicular tumor, endometrium cancer, uterine cervix cancer, ovarian cancer, thyroid cancer, parathyroid gland cancer, nasal cavity cancer, paranasal sinus cancer, skin cancer, malignant melanoma, blood cancer, leukemia, malignant lymphoma, myeloma, angiosarcoma, skin cancer, breast cancer or brain tumor.

Further, examples of the lung cancers include large cell lung cancer, squamous cell lung cancer, small cell lung cancer (SCLC) and non-small cell lung cancer (NSCLC).

The cancer cells may be from an established cancer cell line. Alternatively, the cancer cells are primary cancer cells obtained from a cancer subject/ cancer patient. In some examples, the cancer cells are lung cancer cells, oral cancer cells, fibrosarcoma cells, salivary gland cancer cells, tongue cancer cells, pharyngeal cancer cells, esophagus cancer cells, stomach cancer cells, pancreatic cancer cells, liver cancer cells, bile duct cancer cells, duodenal cancer cells, small intestine cancer cells, large bowel cancer cells, colon cancer cells, rectal cancer cells, renal cancer cells, bladder cancer cells, prostatic cancer cells, penile cancer cells, testicular tumor cells, endometrium cancer cells, uterine cervix cancer cells, ovarian cancer cells, thyroid cancer cells, parathyroid gland cancer cells, nasal cavity cancer cells, paranasal sinus cancer cells, skin cancer cells, malignant melanoma cells, blood cancer cells, leukemia cells, malignant lymphoma, myeloma cells, angiosarcoma cells, skin cancer cells, breast cancer cells or brain tumor cells.

In one example, the lung cancer cells include large cell lung cancer cells, squamous cell lung cancer cells, small cell lung cancer (SCLC) cells and non-small cell lung cancer (NSCLC) cells.

A “pharmaceutical composition” is a formulation containing “the ICD peptides” of the present invention in a form suitable for administration to a subject.

When the pharmaceutical composition of the present invention is used as a medicinal drug, various types of dosage forms can be selected depending upon the administration route.

The dose of the pharmaceutical composition of the present invention is appropriately determined depending upon a purpose for therapy or prophylaxis, and conditions such as sexuality, age, weight of a test subject, an administration route, and degree of a disease.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

All the statistical data represent the average of three independent experiments with standard deviation (mean ± SD) and each experiment set consisting of three replications. The animal survival curves (Kaplan-Meier plot) were analyzed using a log-rank (Mantel-Cox) test. Evaluation of statistical analyses were calculated using one-way ANOVA and Student’s t-tests using GraphPad Prism version 8.0 (San Diego, CA, United States). Significance differences were represented with thresholds of *P<0.05; **P<0.01; ***P<0.001.

### Example 1

### An ICD Peptide Preparation

SEQ ID NO: 1 and its derivatives peptides were artificially synthesised. The molecular weight of the peptides was determined using mass spectrometry and the purities of the peptides (>95%) were assessed by high performance liquid chromatography (HPLC). The sequence of the synthesised SEQ ID NO: 1 and its derivatives peptides as ICD peptides (“ICD” means “immunogenic cell death”) in the present invention was shown as below:

SEQ ID NO: 1 :
Ac-AKR His His GYKRKF His-NH₂

SEQ ID NO: 2 :
Ac-AKR Phe Phe GYKRKF Phe-NH₂

SEQ ID NO: 3 (Nal-P-113):
Ac-AKR Nal Nal GYKRKFNal-NH₂

SEQ ID NO: 4 (Bip-P-113):
Ac-AKR Bip Bip GYKRKF Bip-NH₂

SEQ ID NO: 5 (Dip-P-113):
Ac-AKR Dip Dip GYKRKFDip-NH₂

In one embodiment, His: Histidine; Phe: Phenylalanine; Nal: β-Naphthylalanine; Bip: β-(4.4′-biphenyl)alanine; Dip: β-diphenylalanine.

In other word, the sequence of SEQ ID NO: 1 is Ac-AKRHHGYKRKFH-NH₂ in the present invention.

In another embodiment, N-terminally acylated and C-terminally amidated in sequences of SEQ ID NO: 1, SEQ ID NO: 2, SEQ ID NO: 3, SEQ ID NO: 4, and SEQ ID NO: 5.

TABLE 1

Sequences of SEQ ID NO: 1, SEQ ID NO: 2, SEQ ID NO: 3, SEQ ID NO: 4, and SEQ ID NO: 5.

| SEQ No. | Sequence | Molecular weight (Da) |
|---|---|---|
| SEQ ID NO: 1 | Ac-AKR His His GYKRKF His-NH₂ | 1605.86 |
| SEQ ID NO: 2 | Ac-AKR PhePhe GYKRKF Phe-NH₂ | 1635.99 |
| SEQ ID NO: 3 | Ac-AKR NalNal GYKRKF Nal-NH₂ | 1786.03 |
| SEQ ID NO: 4 | Ac-AKR BipBip GYKRKF Bip-NH₂ | 1864.97 |
| SEQ ID NO: 5 | Ac-AKR Dip Dip GYKRKFDip-NH₂ | 1864.06 |

TABLE 2

Structural properties of aromatic amino acid side chains.

| Amino acid | Phe | Nal | Bip | Dip |
|---|---|---|---|---|
| Chemical structure | 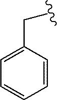 | 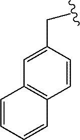 | 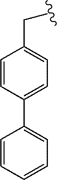 | 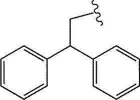 |
| Volume(Å³) | 100.0 | 142.6 | 172.3 | 172.4 |
| Area(Å²) | 274.6 | 430.0 | 391.8 | 387.9 |
| Length(Å) | 4.341 | 6.476 | 8.695 | 4.343 |
| Width(Å) | 2.429 | 4.973 | 2.429 | 7.119 |

### Example 2

### Cell Lines and Culture

Three non-small cell lung cancer cell lines (H1975, A549, PC9) and two oral cancer cell lines (CGNHC9, OECM-1) were used in the present invention. H1975 is the non-small cell lung carcinoma tissue, A549 is the adenocarcinomic alveolar basal epithelial cells and PC9 is the non-small cell lung cancer with EGFR mutation. CGNHC9 and OECM-1 cell lines refer to oral squamous cell carcinoma and gingival epidermoid carcinoma respectively. The abovemetioned cell lines were derived from human origin and being cultured in the humidified incubator containing 5% CO₂ at 37° C. CGNHC9 cell line was cultured in Dulbecco’s modified Eagle’s medium (GIBCO Life Technologies Corporation, NY, USA) with 10% fetal bovine serum (FBS), 2 mM L-glutamine and 1% penicillin/ streptomycin and Roswell Park Memorial Institute 1640 (RPMI 1640, GIBCO Life Technologies Corporation, NY, USA) medium with 10% fetal bovine serum (FBS), 2 mM L-glutamine and 1% penicillin/ streptomycin was used as the culture medium for the rest of the cell lines.

Another, MN-11 cells (murine fibrosarcoma) were cultured in Dulbecco’s modified Eagle medium (DMEM) medium containing 10% FBS, 2 mM L-glutamine and 1% Penicillin/Streptomycin, and incubated in a humidified incubator containing 5% CO2 at 37° C.

On the other side, human diploid fibroblast (HFW) were cultured in DMEM medium supplemented with 10% fetal bovine serum (FBS) and antibiotic. HFW were also cultured in a humidified incubator containing 5% CO₂ at 37° C.

### Example 3

### Cell Viability Determination

The anticancer activity of ICD peptides include, not limited, SEQ ID NO: 1 and its derivative peptides (SEQ ID NO: 2, SEQ ID NO: 3, SEQ ID NO: 4, and SEQ ID NO: 5) was using the 3-(4,5-Dimethyl-2-thiazolyl)-2,5-diphenyl-2H-tetrazolium bromide (MTT, Sigma Aldrich, St. Louis, MO, USA) colorimetric assay for cell viability test. The cells include, not limited, H1975, A549, PC9, CGNHC9, OECM-1, MN-11 and HFW cells/cell lines.

The cells were seeded in the 96-well plate at 5000 cells/well under the environment of humidified 5% CO₂ and the temperature of 37° C. for 24 hours. The medium was removed from the well before the fresh medium containing different concentrations of ICD peptides was added and incubated for 24 hours. Medium with PBS solution was used as control group. All ICD peptides were diluted serially from 100 µM to 0.78 µM in dd_{H2}O before adding in the medium. After 24 hours of ICD peptide treatment, fresh medium with 10% MTT solution (5 mg/mL) was incubated for additional 4 hours in advance of dissolving the formazan crystal in 100 µL DMSO (Dimethyl sulfoxide, Sigma Aldrich) solution. The calculation of cell survival rate was completed by measuring the absorbance at 570 nm using microplate reader (TECAN Sunrise ELISA Reader).

The cell survival was calculated from the treated cells relative to the control (100% viable cells) using the mean of three independent experiments and expressed as a 50% inhibitory concentration (IC₅₀).

The percentage of cell viability was calculated using the following formula:

$$\text{Cell viability \% =} \frac{\text{Absorbance of sample - Blank}}{\text{Absorbance of control - Blank}} \times 100 \%$$

Except SEQ ID NO: 5 peptide having the similar cell survival rate with SEQ ID NO: 2 peptide for A549 cell line, SEQ ID NO: 3, SEQ ID NO: 4 and SEQ ID NO: 5 peptides exhibited the significant anticancer activities in PC9, H1975, CGNHC9 and OECM-1 cell lines. Besides, SEQ ID NO:3, SEQ ID NO:4 and SEQ ID NO: 5 peptides displayed the remarkable anticancer effect by having the cell viability percentage of almost lower than 20% for PC9, OECM-1 and CGNHC9 cell lines. Amidst the three peptides showing outstanding anti-tumour activity, SEQ ID NO:3 demonstrates the lowest cell survival rate in all cell lines tested followed by SEQ ID NO:4 peptide.

On the other side, the result had shown that SEQ ID NO: 1 and SEQ ID NO:2 peptides also employed anticancer activity for CGNHC9 cell line. Unfortunately, SEQ ID NO: 1 and SEQ ID NO:2 peptides have the highest survival rates among every cell line tested which is more than 60% even if the peptide concentration had reached 100 µM.

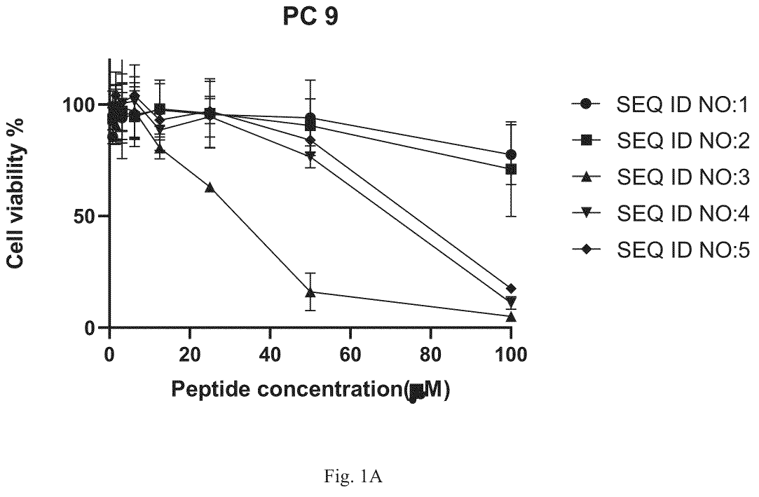

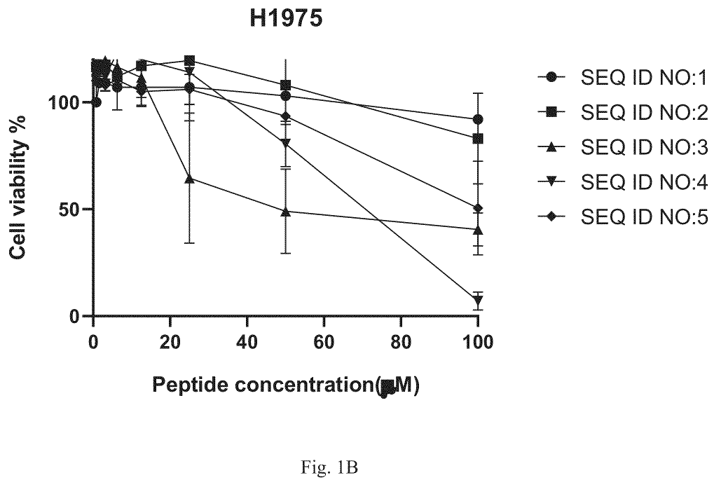

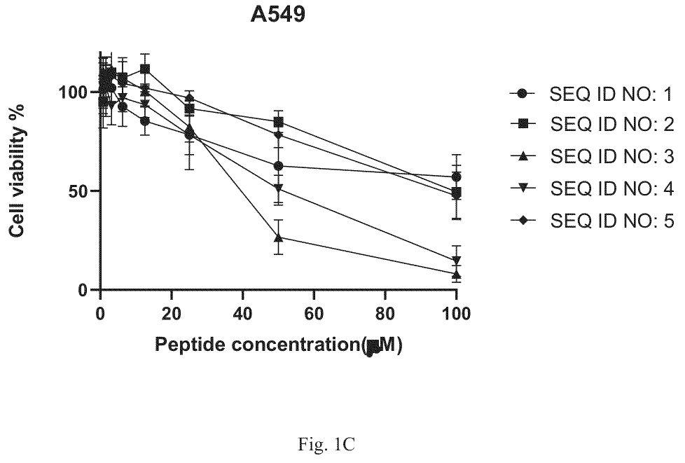

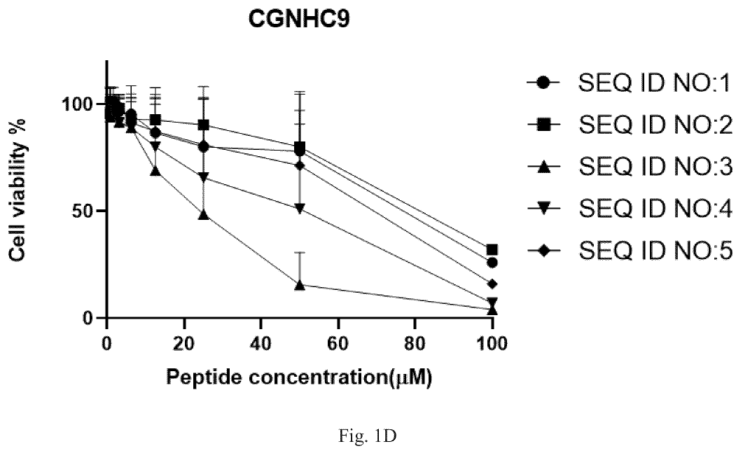

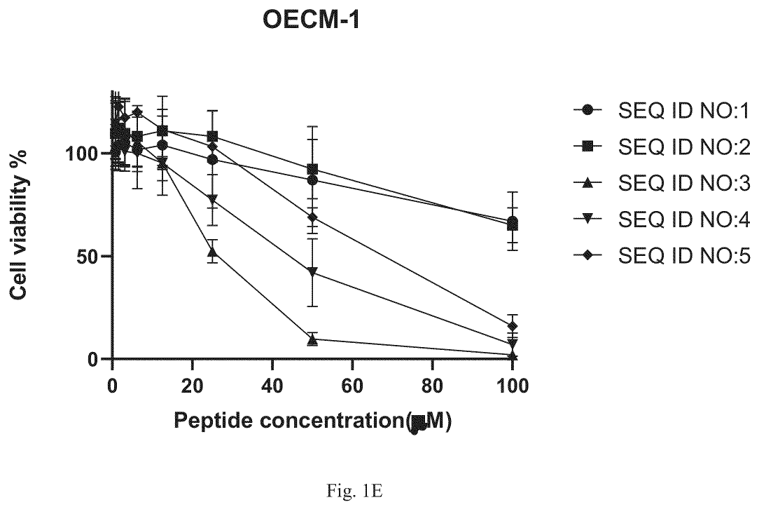

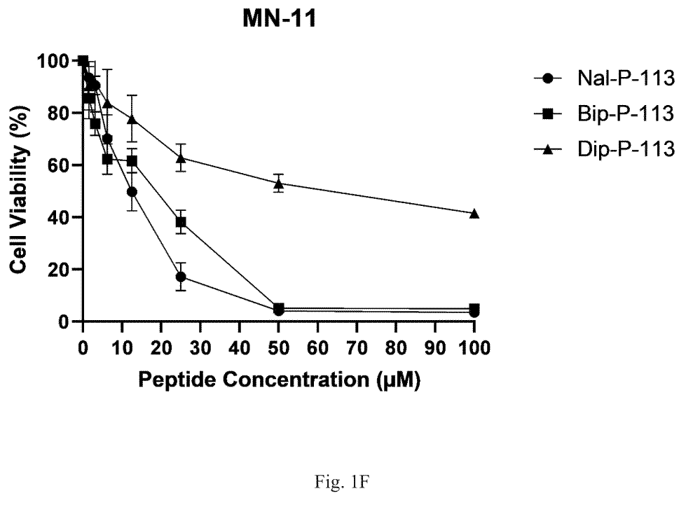

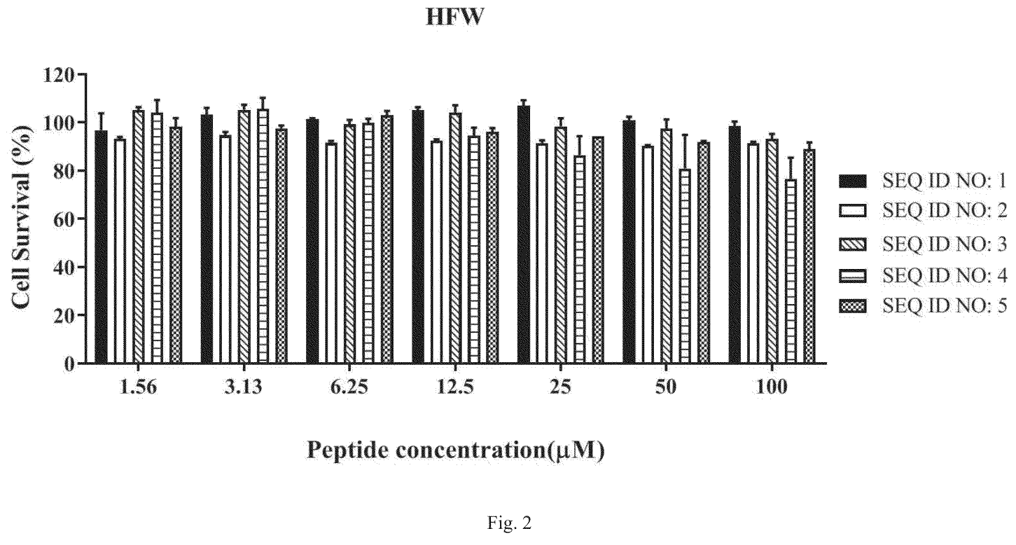

Besides, MTT assay was used to test the cell viability of the murine fibrosarcoma MN-11 cell line. Among the peptides, Dip-P-113 had the lowest cytotoxicity against MN-11 cells (FIG. 1F). Nal-P-113 and Bip-P-113 demonstrated significant anticancer activities against MN-11 cells (FIG. 1F). IC50 of Nal-P-113, Bip-P-113, and Dip-P-113 are 11.12 ± 1.37 µM, 12.13 ± 0.47 µM, and 59.81 ± 6.13 µM, respectively.

However, the results indicated that Nal-P-113, Bip-P-113, and Dip-P-113 only caused less than 10% cell death even at 100 µg/ml.

### Example 4

### Determination of Half Inhibitory Concentration

The half inhibition concentration (IC₅₀) is the concentration required by the drug to reach fifty percent inhibition. In one embodiment, the SEQ ID NO: 1 and its derivative peptides (SEQ ID NO:2, SEQ ID NO:3, SEQ ID NO:4, SEQ ID NO: 5) was used for IC₅₀ test via MTT colorimetric assay. As shown in Table 4, SEQ ID NO: 1 and SEQ ID NO:2 peptide show ineffective effect to inhibit growth of cancer cells/ cancer cell lines.

On the contrary, the other three derivative peptides of SEQ ID NO: 1 have significant anti-tumour activity compared to both SEQ ID NO: 1 and SEQ ID NO:2 peptides, which the SEQ ID NO: 5 peptide has weaker anti-tumour activity among them. The ranges of IC₅₀ values for these ICD peptides administrated to PC9 cell lines varied from 28.11 to 5673 µM.

Table 3. Half inhibitory concentration of SEQ ID NO:1 and its derivative peptides were administrated to cancerous cells/ cancerous cell lines.

| IC₅₀(µM) | SEQ ID NO: 1 | SEQ ID NO:2 | SEQ ID NO:3 | SEQ ID NO:4 | SEQ ID NO: 5 |
|---|---|---|---|---|---|
| Cell line | | | | | |
| H1975 | 110.0 | 106.6 | 58.32 | 63.65 | 100.4 |
| A549 | 106.6 | 99.06 | 38.22 | 48.43 | 96.43 |
| CGNHC9 | 84.27 | 80.49 | 21.37 | 41.97 | 67.89 |
| OECM-1 | 140.6 | 119.9 | 25.91 | 42.47 | 62.94 |
| PC 9 | NA | 172.7 | 28.11 | 64.19 | 71.45 |

### Example 5

### Kinetic Analysis for Cancer Cells Via Administrating the ICD Peptides

The killing kinetics of the derivative peptides of SEQ ID NO: 1 which including SEQ ID NO:3, SEQ ID NO:4 and SEQ ID NO: 5 were carried out on the cancer cells/ cancer cell lines using the calculated 2 × IC₅₀ concentration. The method of cell seeding was the same as the method previously described in the cell viability determination for MTT assay which is 5 × 10³ cells/mL.

The cells including, not limited PC9 cells, were incubated with the three of the peptides which derived from the ICD peptide, SEQ ID NO: 1 for the time points of 0.5, 1, 2, 3 and 4 hours. The further incubation of the MTT solution for another 3 hours was carried out immediately after the final time point. Absorbance was measured at 570 nm after the solubilisation of the formazan crystal in 100 µL DMSO solution. Lastly, cell survival rate was calculated as aforementioned for cell viability assay.

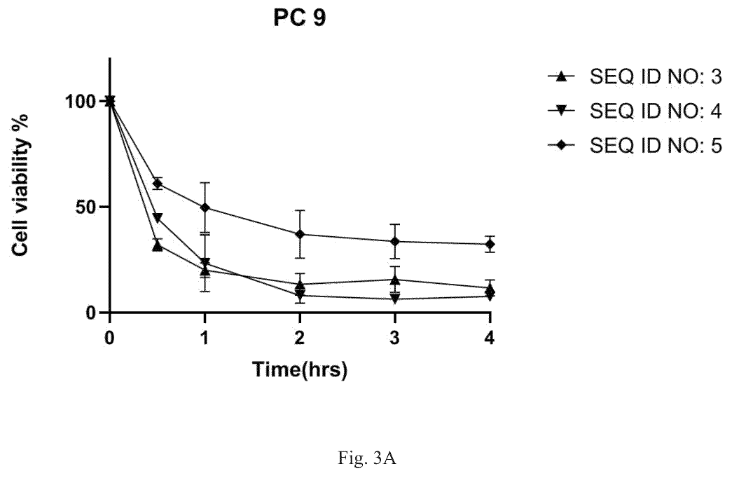

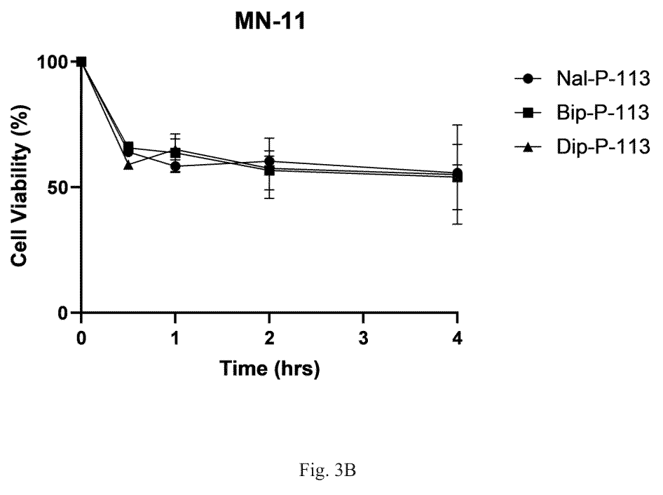

The two-fold half inhibitory concentration was chosen for three independent experiments and the results were recorded from 30 minutes to 4 hours. As shown in the FIG. 3A, SEQ ID NO:3 and SEQ ID NO:4 had started to kill the cells after 30 minutes of ICD peptides incubation and thus reduced the cell survival rate to beyond 50%. The data showed that these three derivative peptides have no significant change in survival rate between 2 to 4 hours implementing the rapid action of the ICD peptides toward the cancer cells. Within 2 hours, SEQ ID NO:3 and SEQ ID NO:4 had effectively killed more than 80% of the cells making both of the peptides the most effective peptide for the anticancer activity.

On the other hand, the cells treated with SEQ ID NO: 5 had the highest cellular survival rate at around 60% in 30 minutes. The overall cell survival rate of the group treated with SEQ ID NO: 5 was the highest compared to SEQ ID NO:3 and SEQ ID NO:4 group.

Besides, evaluation of the killing kinetics of Nal-P-113, Bip-P-113 and Dip-P-113 for MN-11 was determined by MTT assay. Two-fold half 1inhibitory concentration (2 × IC₅₀) was chosen for the experiments and the results were recorded at selected time points (30 mins, 1 hour, 2 hours and 4 hours). As shown in FIG. 3B, these three peptides started to kill cancer cells within 30 minutes of peptide treatment. In 2 hours, the cell survival rate was near 50%.

### Example 6

### Detection of Extracellular HMGB1 From Cancer Cells Administrated With the ICD Peptides

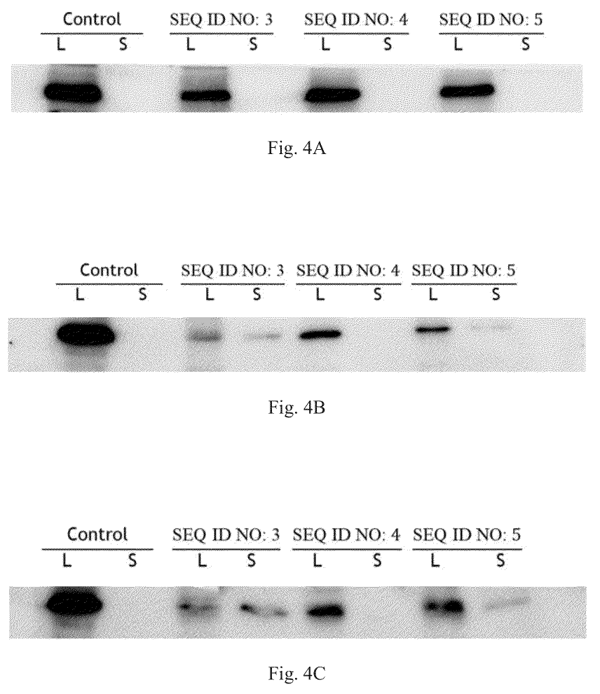

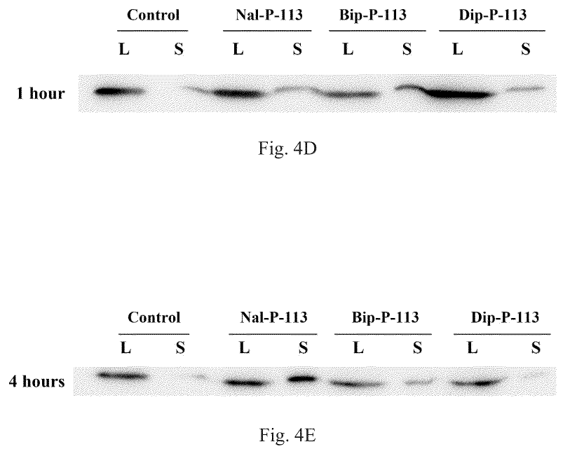

HMGB 1 is a chromatin-binding protein and plays an important role as an indicator in the peptide induced immunogenic cell death. In one embodiment, the PC 9 cells were seeded at the density of 2 × 10⁴ cells/well under the culture condition in 6-well plate as mentioned earlier in the cell viability determination and left to adhere for 48 hrs. The cells were treated with 56.22 µM of SEQ ID NO:3, 128.38 µM of SEQ ID NO:4 and 142.9 µM of SEQ ID NO: 5 peptides which is the two-fold IC₅₀ concentration for 1 hour (FIG. 4A), 2 hour (FIG. 4B), 4 hour (FIG. 4C) under the incubation condition of humidified 5% CO₂ and 37° C. The ICD peptides used were dissolved thoroughly in the serum-free medium (RPMI 1640) before treating the cells.

After the treatment of ICD peptides, the supernatant and lysate of the cells were collected separately for the further experiment. The supernatant was centrifuged at the speed of 1,500 RCF while the cell lysate was undergoing the centrifugation at 14,000 rpm for 5 minutes under the temperature of 4° C. Then, the pellet collected from the supernatant and supernatant of centrifuged cell lysate were discarded. The harvested pellet of cell lysate was washed twice using 200 µL PBS each time preliminarily the sonication of the pellet using bio-disruptor. The disrupted pellet was centrifuged again for 10 minutes under 14,000 rpm and 4° C.

The concentration of the samples was quantified using the commercially available protein quantification assay such as Bradford assay to make sure the amount of the protein was equally loaded in each of the well for SDS-PAGE gel.

The transfer process from polyacrylamide gel to PVDF membrane was performing for 75 minutes under 30 V and 400 mA as well as semi-dry condition. PBST buffer was used to wash the PVDF membrane three times after the transfer process.

The anti-HMGB1 antibody (Abcam, Cambridge, UK, ab18256) will be the primary antibody and incubated overnight at 4° C. in the dilution ratio of 1: 1000. The secondary antibody of rabbit (diluted in 1: 10000) was incubated following the rinsing with PBST three times for 2 hours the next day in the room temperature before being developed using luminol reagent and imaged by ImageQuant LAS 4000.

The HMGB 1 protein band of nearly 29 kDa for both cell lysate and supernatant as early as 2 hours of treatment with the ICD peptides SEQ ID NO:3, SEQ ID NO:4 and SEQ ID NO: 5. As shown in FIGS. 4A-4C, the PC 9 cells treated with only serum free RPMI 1640 medium act as control group for this Western Blot analysis and nothing is observed in the supernatant and this appeared to be same as 1 hour treatment group but HMGB 1 can clearly be seen in the supernatant for 2 hour and 4 hour treatment group.

In contradictory, HMGB 1 can be detected in the supernatant (represented by “S”) after the treatment with SEQ ID NO:3 and SEQ ID NO: 5 for 2 hours but the supernatant of 2 hours treated SEQ ID NO:4 can hardly detected the HMGB 1 protein. Comparing both 2 hour and 4 hour treatment group, the HMGB 1 protein was release from cell lysate (represented by “L”) to supernatant and the band was obviously be seen in supernatant of 4 hours group while the lysate band was reduced and faded in the same time for SEQ ID NO:3 and SEQ ID NO: 5. Although HMGB1 is not detectable in the supernatant for both 2 hour and 4 hour, the cell lysate band for 4 hours group was started to fade, suggesting that SEQ ID NO:4 might undergo apoptosis pathway while SEQ ID NO:3 and SEQ ID NO: 5 vitally depending on the secondary necrosis.

On the other hand, The MN-11 cells were seeded at the density of 1 × 10⁵ cells/well and left to adhere for 24 hrs. Cells were treated with Nal-P-113, Bip-P-113 and Dip-P-113 at 2× IC₅₀ for 1 and 4 hours. After treatment of the peptides, the supernatant and lysate of the cells were collected separately for further experiments. The supernatant was collected after centrifugation at the speed of 1,400 ×g. The cell lysate was washed twice using PBS before the sonication of the pellet using bio-disruptor, and the disrupted pellet was centrifuged again for 10 minutes under 14,000 rpm at 4° C. The concentration of the samples was quantified using the Bradford assay. Both supernatants and cell lysate were loaded and run on the sodium dodecyl sulfate polyacrylamide gel electrophoresis (SDS-PAGE), and then electro transferred to a polyvinylidene difluoride (PVDF) membrane (Merck-Millipore, Burlington, Massachusetts, United States) in electro-blot system, 30 V, 400 mA, for 75 mins. The membrane was incubated in blocking buffer (5% skim milk, TBST buffer) for 1 hour in room temperature and washed in TBST buffer three times. The anti-HMGB 1 antibody (Abcam, Cambridge, UK, ab18256) will be the primary antibody and incubated overnight at 4° C. in the dilution ratio of 1: 1000. The secondary antibody of rabbit (diluted in 1: 10000) was incubated following the rinsing with PBST three times for 2 hours the next day in the room temperature before being developed using luminol reagent and imaged by a detected system (ImageQuant LAS 4000 mini).

The protein band of HMGB 1 (29 kDa) for both cell lysate and supernatant was used to detect HMGB1 protein release after treatment with Nal-P-113, Bip-P-113 and Dip-P-113. As shown in FIG. 4D, HMGB 1 was observed in the supernatant following 1 hour of treatment with Nal-P-113, Bip-P-113 and Dip-P-113. There is still a significant concentration of HMGB 1 in the supernatant even after 4 hours in the Nal-P-113 treatment group (FIG. 4E). On the other hand, HMGB 1 in the supernatant decreased rapidly after 1 hour and could be hardly detected in the Dip-P-113 treatment group.

### Example 7

### Analyse ROS Release From Cancer Cells Administrated With the ICD Peptides

Release of reactive oxygen species (ROS) would be come from dysfunctional mitochondria as an important key in immunogenic cell death. That is to say that reactive oxygen species (ROS) production may be caused by dysfunctional mitochondria. 2′,7′-dichlorofluorescin diacetate (DCFDA) Cellular ROS Detection Assay Kit (Abcam, Cambridge, UK, ab113851) was used to analyse the ROS release in the PC 9 cell line via administrated with ICD peptides.

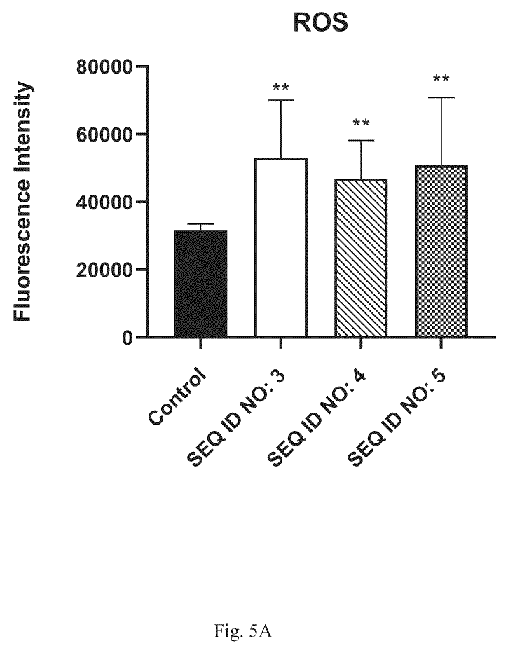

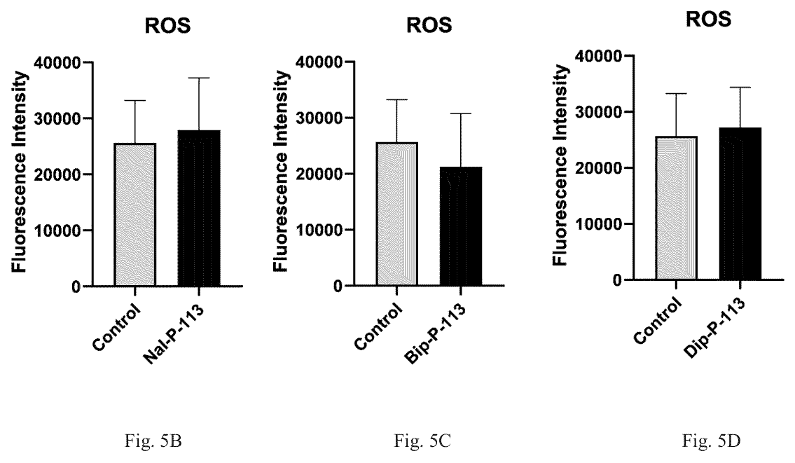

25,000 cells/ well of the PC 9 cells were seeded in the black-sided 96 well plate with clear bottom and let it to adhere for 24 hours prior the experiment. 100 µL/ well of 1x buffer was applied for washing purpose to remove the RPMI 1640 medium. Therefore, 100 µL of the diluted DCFDA solution was added into the well after removing the 1x buffer solution and incubated for 45 minutes at 37° C. in the dark condition to stain the cells. 100 µL/ well of 1x buffer in 1x PBS solution was applied after the removal of diluted DCFDA solution. The SEQ ID NO:3, SEQ ID NO:4 and SEQ ID NO: 5 peptides of 2 × IC₅₀ concentration were used to treat the PC 9 cells for 1 hour, 2 hour and 4 hour respectively. TBHP diluted in 1x buffer solution served as positive control while untreated cells will be the negative control. The black-sided 96 well plate was undergoing the measurement instantly using the fluorescence plate reader, Wallac VICTOR 3 Multilabel plate reader (PerkinElmer, Shelton, CT, USA) at the Ex/Em = 485/535 nm in the presence of peptides solution (FIG. 5A).

However, MN-11 cells were seeded at the density of 25,000 cells/well in the 96-well black plate and allowed to adhere for 24 hours. Cells were washed with a 100 µL/well of PBS to remove the RPMI 1640 medium, and incubated with 100 µL of the 25 µM DCFDA solution for 45 minutes at 37° C. in the dark condition. 100 µL/ well of PBS buffer was washed again for the removal of 25 µM DCFDA solution. The MN-11 cells were treated with Nal-P-113, Bip-P-113 and Dip-P-113 at 2× IC50 for 2 hours. Cells incubated with DMEM without phenol red was used as negative control, and the positive control was tertbutyl hydroperoxide (TBHP) diluted in the 1x Supplemented Buffer. The fluorescence intensity of cellular ROS was determined to an excitation wavelength of 485 nm and an emission wavelength of 535 nm on the Wallac VICTOR 3 Multilabel plate reader (PerkinElmer, Shelton, CT, USA). FIGS. 5B-5D indicates that fluorescence intensity did not change even after a 2-hour treatment on MN-11 cells with 2 × IC₅₀ of Nal-P-113, Bip-P-113 and Dip-P-113. These results indicated that Nal-P-113, Bip-P-113 and Dip-P-113 did not promote ROS production of MN-11 cells.

### Example 8

### Luciferin-Luciferase Detection for Extracellular ATP Releasing From Cancer Cells Administrated With the ICD Peptides

The ATP release from cancer cells were measured using the luciferin-luciferase assay as ATP release had been the iconic feature for immunogenic cell death (ICD).

PC 9 cells were seeded in the 96 well plate at the density of 10,000 cells/ well and allowed to adhere for 48 hours in the condition described previously for MTT assay. The ICD peptides (SEQ ID NO:3, SEQ ID NO:4 and SEQ ID NO: 5) were used to treat the PC 9 cells after the incubation at the concentration of 2 × IC₅₀ concentration for 0.5, 1, 1.5 and 2 hours respectively. All of the three ICD peptides were dissolved in the serum-free RPMI 1640 medium for the cells’ treatment. Cells were treated with serum-free RPMI 1640 medium containing PBS as the control group. After being treated for 0.5, 1, 1.5, and 2 hours, the supernatant of cells was collected separately and stored at -20° C. before the ATP assay. The aliquot of reconstituted rL/L reagent in the ENLITEN ATP assay kit (Promega, Madison, WI, USA) was incubated at room temperature for one hour before using. The collected supernatant of the cells was centrifuged at the speed of 500 xg for 10 minutes and took out 100 µL of the supernatant into the black 96 well plate with clear bottom to avoid cross-talk between wells. As the luciferase reaction will begin immediately upon addition of the reconstituted rL/L reagent into the samples, the row of samples was processed individually by adding the rL/L reagent and began the light output measurement. Measurement of the extracellular ATP chemiluminescence was completed using the Wallac VICTOR 3 Multilabel plate reader (PerkinElmer, Shelton, CT, USA)

The relative light unit recorded the highest value for all three ICD peptides at the treatment time of 0.5 hour and declining over the time. Although SEQ ID NO: 3, SEQ ID NO: 4, SEQ ID NO: 5 have been observed to reach the peak at 0.5-hour time point, SEQ ID NO: 4 peptide has relatively lower ATP release among them while SEQ ID NO: 3 and SEQ ID NO: 5 have similar amount of bioluminescence release at around 10,000 RLU.

Compared to both SEQ ID NO: 3 and SEQ ID NO: 5 peptides, the highest value of ATP release of SEQ ID NO: 4 was lower than 5,000 RLU and the lowest value recorded was around 1,500 RLU at 2 hours. The group of PC 9 cells treated with SEQ ID NO: 3 and SEQ ID NO: 5 peptide have greatest ATP release in supernatant after incubated for 0.5 hour and the ATP amount detected in the supernatant were decrease gradually over time. The same pattern of data can also be observed at 1, 1.5 and 2 hours respectively.

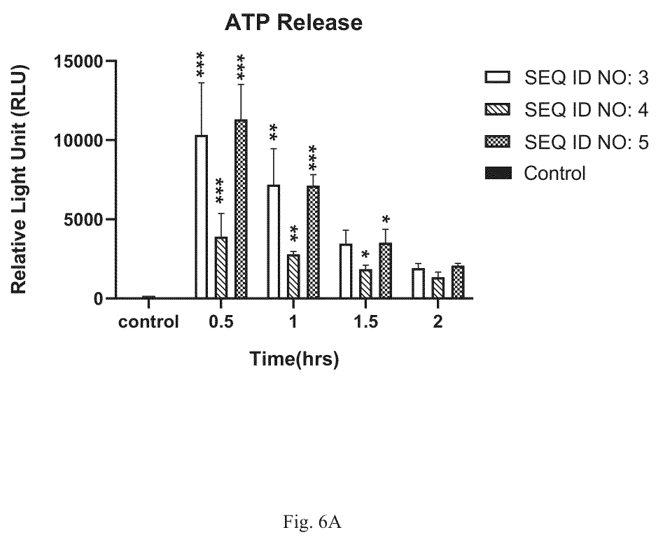

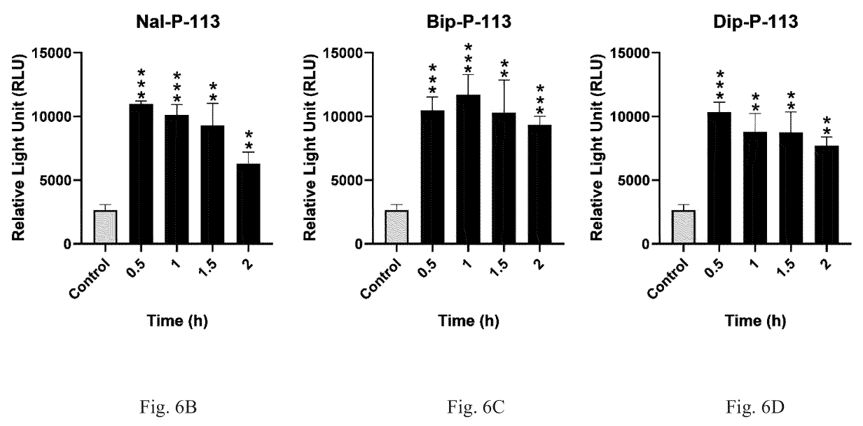

In conclusion, the release of ATP was decrease in the matter of time for SEQ ID NO:3, SEQ ID NO:4 and SEQ ID NO: 5 peptide but having an obvious change in the ATP level after the treatment comparing to control group. The data of luciferin-luciferase assay were expressed in FIG. 6A.

On the other hand, MN-11 cells were seeded at the density of 25,000 cells/ well in the 96 well plate and allowed to adhere for 24 hours. Cells were treated with Nal-P-113, Bip-P-113 and Dip-P-113 at 2× IC₅₀ for 0.5, 1, 1.5, and 2 hours. Untreated cells served as the control group. After being treated for different time points (0.5, 1, 1.5, and 2 hours), the supernatant of cells was analyzed using the ENLITEN ATP luciferase assay kit (Promega, Madison, WI, USA). The level of the extracellular ATP chemiluminescence was measured at 560 nm using the Wallac VICTOR 3 Multilabel plate reader (PerkinElmer, Shelton, CT, USA).

ATP release from MN-11 cells were measured using the luciferin-luciferase assay after peptide (2 × IC₅₀) treatment (FIGS. 6B-6D). Nal-P-113 and Dip-P-113 reached the relative light unit (~ 11,000 RLU) peak at 0.5-hour, and Bip-P-113 reached the highest peak at 1 hour (~ 13,000 RLU). Nevertheless, Nal-P-113 (FIG. 6B), Dip-P-113 (FIG. 6C), and Bip-P-113 (FIG. 6D) all display similar amounts of ATP release.

### Example 9

### Detection of Extracellular Cytochrome C From Cancer Cells Administrated With the ICD Peptides

The cytochrome c release was a part of mitochondrial damage-associated molecular patterns, therefore the amount of cytochrome c released into supernatant were measured by cytochrome c ELISA assay.

The cells were washed three times using PBS solution to remove all serum components and 1× 10⁶ cells/ mL was solubilised in ice-cold 0.5% Triton-X 100-PBS solution at 2-8° C. for 10 minutes. Then, the speed of 16,000 xg was applied to centrifuge the extract for another 10 minutes, and serially dilute the aliquot of supernatant collected with Calibrator Diluent RD5-18 as described in the manufacturer’s protocol. The density of 10,000 cells/ well was seeded in the 96 well plate for 48 hours in addition to treat the seeded cells with 2-fold IC₅₀ of SEQ ID NO:3, SEQ ID NO:4 and SEQ ID NO: 5 which is 56.22 µM, 128.38 µM and 142.9 µM respectively preceding the procedure of detecting the extracellular Cytochrome c using the Quantikine ELISA Rat/ Mouse Cytochrome c Immunoassay (R & D Systems, Minneapolis, MN, USA).

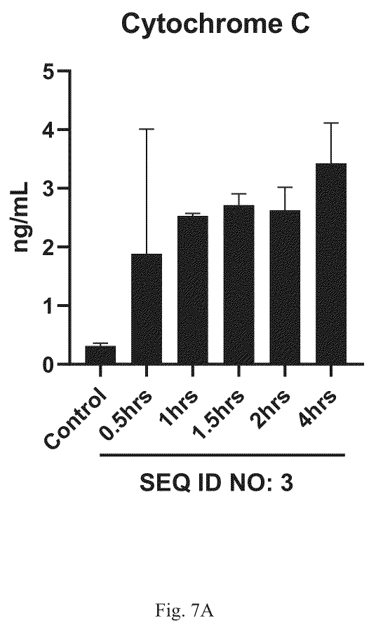

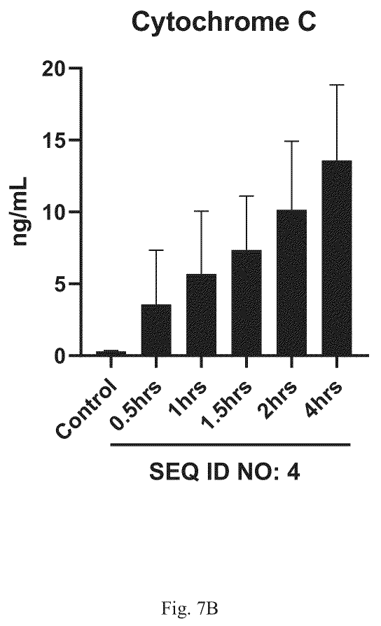

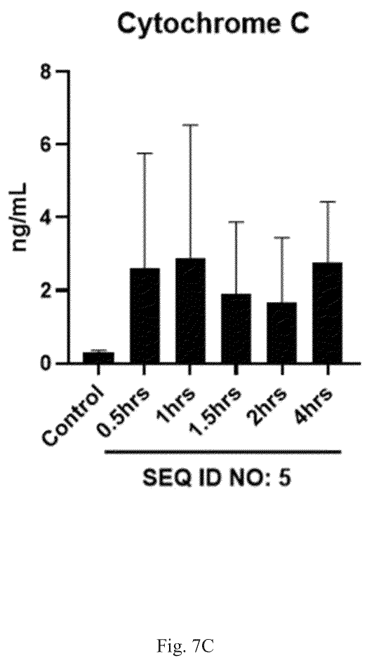

Therefore, the amount of cytochrome c released into supernatant were measured by cytochrome c ELISA assay to investigate the capability of the SEQ ID NO:3, SEQ ID NO:4 and SEQ ID NO: 5 peptide to induce the mitochondrial DAMPs. Quantifying the volume of cytochrome c released and the results were presented in FIGS. 7A-7C via ELISA assay for 0.5, 1, 1.5, and 2 hours. SEQ ID NO:4 group induced a relatively high amount of cytochrome c compared to SEQ ID NO:3 group and SEQ ID NO: 5 group. The cytochrome c release for SEQ ID NO:4 group was double the amount of SEQ ID NO:3 group and SEQ ID NO: 5 group. The trend of cytochrome c release in SEQ ID NO:4 and SEQ ID NO:3 treated PC 9 cells were escalating from 30 minutes to 4 hours, in contrast the SEQ ID NO: 5 group has fluctuating trend. The amount of cytochrome c release has come to the maximal value of nearly 15 ng/mL at 4 hours while the minimum value of 3 ng/mL at 30 minutes of ICD peptide incubation. The highest amount of cytochrome c detected in the supernatant for SEQ ID NO:3 and SEQ ID NO: 5 were around 3.5 ng/mL and lower than 2 ng/mL for the least. A slightly cytochrome c was detected in the control group and it was extremely low compared to the derivative ICD peptides treated cells suggesting the capability of these three ICD peptides to translocate cytochrome c into supernatant and thus triggered the features of immunogenic cell death.

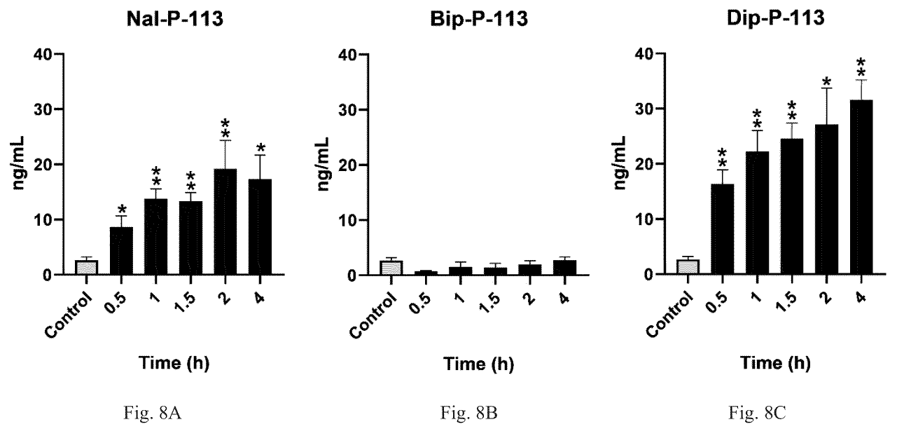

Not only that the amount of supernatant cytochrome c release from MN-11 cells was measured by ELISA assay to reveal peptide induced mitochondrial DAMPs. Five time points (0.5, 1, 1.5, 2 and 4 hours) were used to quantify the amount of cytochrome c released and the results were shown in FIGS. 8A-8C. Nal-P-113 (FIG. 8A) and Dip-P-113 (FIG. 8C) induced a relatively high amount of cytochrome c compared to Bip-P-113 (FIG. 8B). In the Nal-P-113 (FIG. 8A) treated group, the amount of cytochrome c release has come to a maximal value of nearly 25 ng/mL at 2 hours while the minimum value of 10 ng/mL at 30 minutes has been observed. The highest amounts of cytochrome c detected in the supernatant for Dip-P-113 (FIG. 8C) was around 35 ng/mL at 4 hours and the lower amount around 18 ng/mL at 30 minutes. However, only a very low amount (similar to the control group) of cytochrome c was detected in the Bip-P-113 group. These results suggested that Nal-P-113 and Dip-P-113 may cause the release of cytochrome c into the supernatant and thus, triggering the features of immunogenic cell death.

### Example 10

### Loss of Plasma Membrane Integrity by Propidium Iodide (PI)/Hoechst 33342 Staining

To examine the membrane-lytic activities of peptides, the PI/Hoechst 33342 staining was performed in this embodiment. The PI dye was used to stain damaged/dead cells, and Hoechst 33342 was used to specifically stain the nuclei of living cells. MN-11 cells were seeded at 50,000 cells/well in a RPMI medium and allowed to adhere for 24 hours. Then the cells were replaced with serum-free RPMI medium, and then treated with 2× IC₅₀ peptides at 37° C. for 2 hrs. DMEM medium without peptide was used as negative control. The PI and Hoechst 33342 were added at a final concentration of 1 µg/ml for 20 mins. The cells were observed using the inverted fluorescent microscope Zeiss equipped with a 40× oil objective lens (Carl Zeiss, Germany). The experiments were repeated three times independently

To examine the membrane-lytic activities of Nal-P-113, Bip-P-113, and Dip-P-113, the DNA binding fluorescent dye Propidium iodide (PI) was used to stain damaged/dead cells, and Hoechst 33342 was used to specifically stain the nuclei of living cells. The results demonstrated that Nal-P-113, Bip-P-113, and Dip-P-113 can cause damage to the plasma membrane integrity of MN-11 cells and increase the amount of PI entering into MN-11 cells (Data not shown).

### Example 11

### Nal-P-113 Induces Tumor Regression

Female C57BL/6 wild-type mice (4–5 weeks old) were obtained from the BioLASCO Taiwan Co., Ltd (Taiwan). All mice were housed in a specific pathogen-free and controlled environment. During the experiments, female mice, weighing 18–24 g each, were kept in groups of 4 to 6 animals per cage under climate-controlled conditions, with 12 h light/dark cycles and an ambient temperature. The mice were housed in an enriched individually ventilated cage system with free access to standard rodent chow and water ad libitum. The animals were anesthetized during the experimental procedures with 2.5% isoflurane gas. The animals were monitored daily and large-tumor-bearing mice were euthanized with isoflurane. All the procedures were conducted according to the regulations of Laboratory Animal Care and Use Committee or Group Setup and Management and the law of Animal Protection. All animal experiments were approved by National Tsing Hua University (approval ID: 110016), and the protocol was approved by Institutional Animal Care and Use Committee.

Pre-cultured MN-11 cells were harvested in a DMEM medium and inoculated total of 5×10⁵ cells (mixed equal volume of matrigel) into the right flank of the abdomen in C57BL/6 mice with subcutaneous injection (6 mice/group). Palpable tumors (50-75 mm³) were intratumorally injected (i.t.) with Nal-P-113 (1 mg/50 µl saline per mouse) for 3 consecutive days. Negative control (tumor only) mice received saline only (sterile H₂O with 0.9% NaCl). Tumor size was measured every 2 days using a caliper, and the volume of tumors was calculated using the following formula: Tumor size (mm³) = 0.5× (width²×length). Animals were terminated when the tumor exceeded 2000 mm³. The survival rates of tumor-bearing mice were observed and recorded continuously until all animals were dead. Additionally, the following were implemented as humane endpoints: signs of unacceptable pain, infection, severe ulceration, or surgical complications.

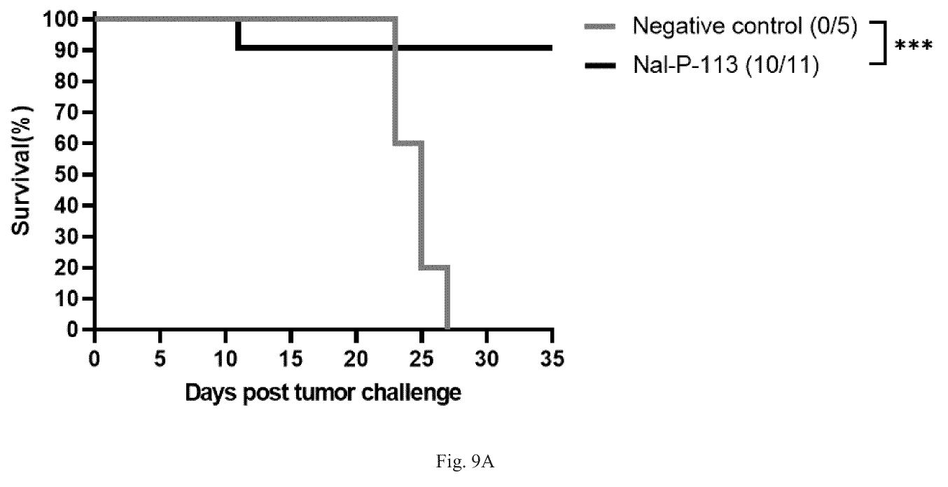

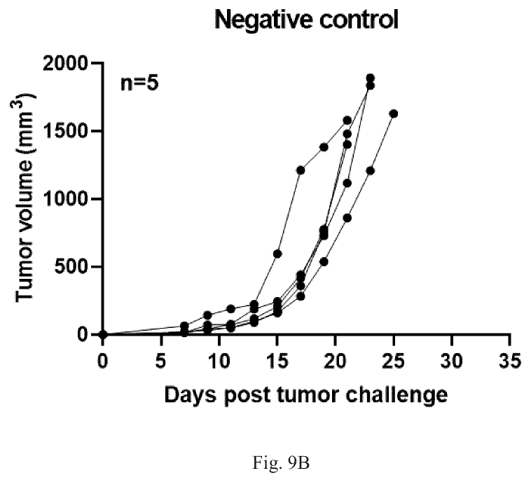

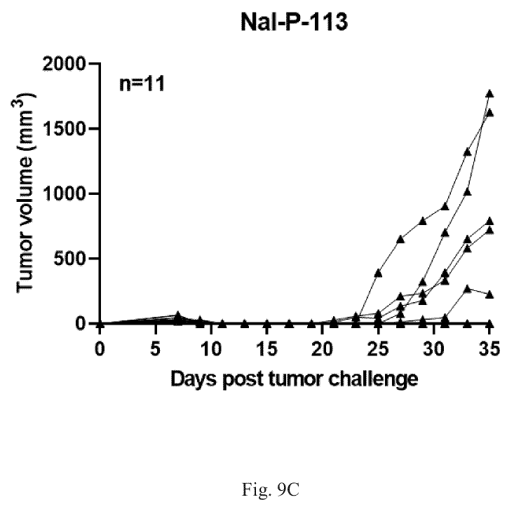

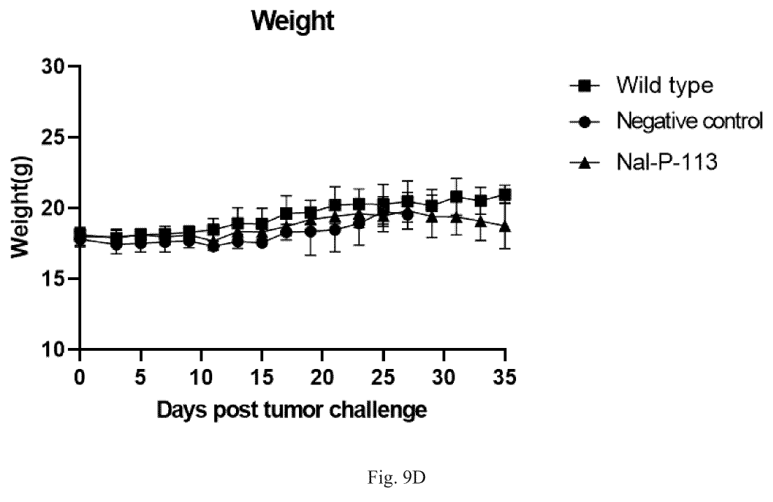

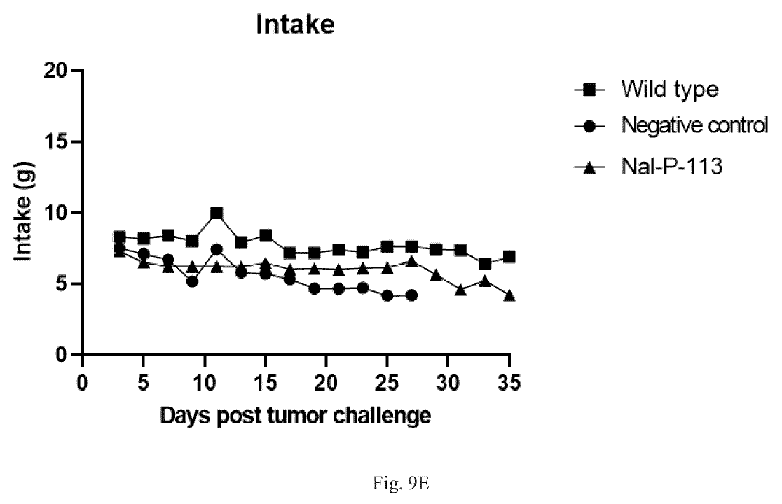

To investigate the therapeutic potential of Nal-P-113 in vivo, MN-11 cells were subcutaneously implanted into C57BL/6 mice. After eight days post-implantation, tumor volumes reached 50-75 mm³. Then all animals were injected intratumorally (i.t.) with 50 µl of PBS (Negative control; n=5) or 1 mg/ml Nal-P-113 (n=11). The results indicated that the Nal-P-113 treatment group showed the highest survival rate while all animals in the control group died within 28 days (FIG. 9A). At the end of the experiments (35 days), 10 out of 11 animals were survived in the treatment group. Complete regression of the tumor was observed in 5 animals and 6 animals had tumor recurrence in the treatment group (FIG. 9C). Nal-P-113 treatment appeared to be nontoxic since no there were effects on the body weight and intake of the animals at the end of the experiment (FIG. 9D and FIG. 9E).

### Example 12

### Tumor Re-Challenge, Then Nal-P-113 Previous Treatment Leads to a Long-Term Antitumor Immunity

Animals with a complete regression of tumor after Nal-P-113 treatment were given a s.c. tumor cell re-challenge (5×10⁵ MN-11 cells) on the left flank of the abdomen in C57BL/6 mice. Tumor size was measured every 2 days using a caliper, and the volume of tumors was calculated using the following formula: Tumor size (mm³) = 0.5×(width²×length). The survival rates of tumor-bearing mice were observed and recorded

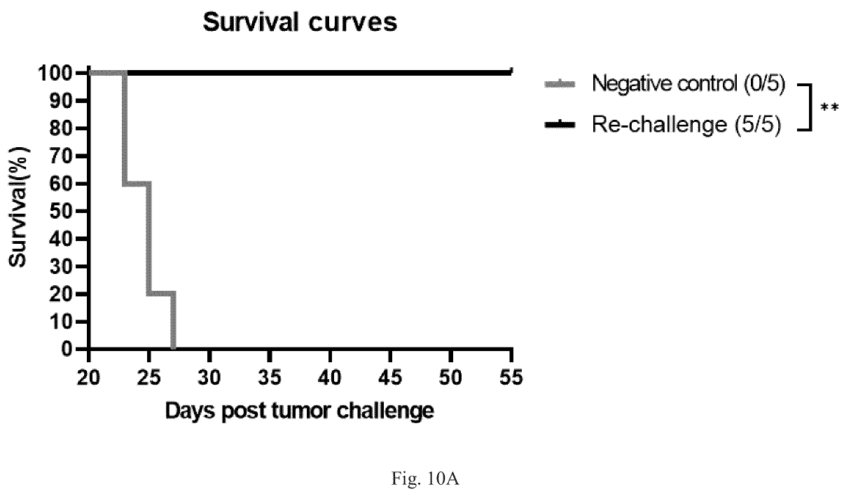

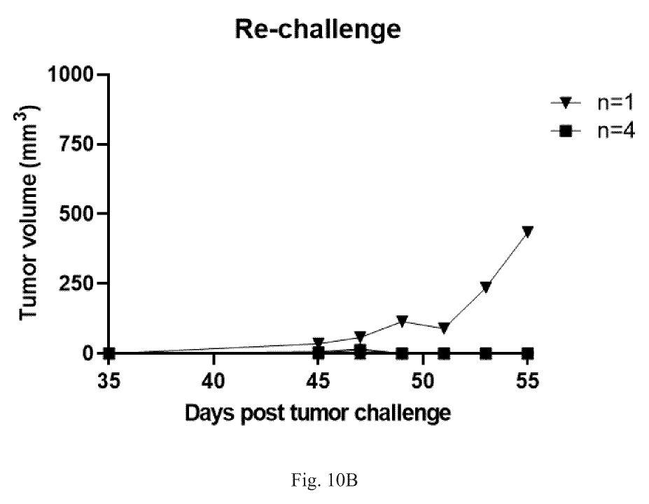

To examine whether the treatment with Nal-P-113 was able to induce long-term antitumor immunity, animals with complete regression were re-challenged with MN-11 cells (Subcutaneous injection; n=5). All animals were protected from the MN-11 cells re-challenge and stayed alive until the end of the experiments (55 days) (FIGS. 10A-10B).

In summary, the present invention demonstrated to identify the anti-tumour activity of SEQ ID NO: 1 and its derivative peptides (SEQ ID NO:2, SEQ ID NO:3, SEQ ID NO:4 and SEQ ID NO: 5) and how they induced the immunogenic cell death. By identifying the danger signals (DAMPs) such as ROS, ATP and cytochrome c release as well as the secretion of nuclear protein HMGB1, SEQ ID NO:3 and SEQ ID NO: 5 demonstrated to undergo the apoptotic cell death pathway at first then converted into necrosis cell death in the very quickly time course while SEQ ID NO:4 peptide carried on the apoptotic cell death and undergone the secondary necrosis and inducing immunogenic cell death via the observation of damage-associated molecular patterns’ secretion based on the embodiments that high level of ROS, ATP and cytochrome c had been secreted into the supernatant

The foregoing is illustrative of the present invention, and is not to be construed as limiting thereof. The invention is defined by the following claims, with equivalents of the claims to be included therein.

What is claimed is:

1. An ICD peptide for use in a method to inhibit cancer cells viability, comprising:
administering the ICD peptide in a therapeutically effective amount to a subject in need thereof;
wherein the ICD peptide selected from the group consisting of SEQ ID NO: 1, SEQ ID NO:2, SEQ ID NO:3, SEQ ID NO:4 and SEQ ID NO:5;
wherein the subject is diagnosed with cancer cells.

2. The ICD peptide for use according to claim 1, wherein the cancer cells comprise lung cancer cells, oral cancer cells, fibrosarcoma cells, salivary gland cancer cells, tongue cancer cells, pharyngeal cancer cells, esophagus cancer cells, stomach cancer cells, pancreatic cancer cells, liver cancer cells, bile duct cancer cells, duodenal cancer cells, small intestine cancer cells, large bowel cancer cells, colon cancer cells, rectal cancer cells, renal cancer cells, bladder cancer cells, prostatic cancer cells, penile cancer cells, testicular tumor cells, endometrium cancer cells, uterine cervix cancer cells, ovarian cancer cells, thyroid cancer cells, parathyroid gland cancer cells, nasal cavity cancer cells, paranasal sinus cancer cells, skin cancer cells, malignant melanoma cells, blood cancer cells, leukemia cells, malignant lymphoma, myeloma cells, angiosarcoma cells, skin cancer cells, breast cancer cells or brain tumor cells.

3. The ICD peptide for use according to claim 2, wherein the lung cancer cells are selected from large cell lung cancer cells, squamous cell lung cancer cells, small cell lung cancer (SCLC) cells, and non-small cell lung cancer (NSCLC) cells.

4. The ICD peptide for use according to claim 2, wherein the ICD peptide can inhibit cancer cells viability, lead to HMGB1 passive release from the cancer cells, lead to release of ATP from the cancer cells, lead to release of cytochrome c, and loss of plasma membrane integrity.

5. A method for inhibiting cancer cell viability and disrupting membrane of cancer cells, comprising:
administering to a subject in need thereof a therapeutically effective amount of a ICD peptide;
wherein the ICD peptide selected from the group consisting of SEQ ID NO: 1, SEQ ID NO:2, SEQ ID NO:3, SEQ ID NO:4 and SEQ ID NO:5;
wherein the subject is diagnosed with cancer.

6. The method according to claim 5, wherein the cancer comprises lung cancer, oral cancer, fibrosarcoma, salivary gland cancer, tongue cancer, pharyngeal cancer, esophagus cancer, stomach cancer, pancreatic cancer, liver cancer, bile duct cancer, duodenal cancer, small intestine cancer, large bowel cancer, colon cancer, rectal cancer, renal cancer, bladder cancer, prostatic cancer, penile cancer, testicular tumor, endometrium cancer, uterine cervix cancer, ovarian cancer, thyroid cancer, parathyroid gland cancer, nasal cavity cancer, paranasal sinus cancer, skin cancer, malignant melanoma, blood cancer, leukemia, malignant lymphoma, myeloma, angiosarcoma, skin cancer, breast cancer or brain tumor.

7. The method according to claim 6, wherein the lung cancer is selected from large cell lung cancer, squamous cell lung cancer, small cell lung cancer (SCLC) and non-small cell lung cancer (NSCLC).


SEQUENCE LISTING (5 sequences)

SEQ ID NO: 1 - protein, 12 aa, synthetic construct
  BINDING 1: note = N-terminal acetylation
  BINDING 12: note = C-terminal amidation
AKRHHGYKRKFH

SEQ ID NO: 2 - protein, 12 aa, synthetic construct
  BINDING 1: note = N-terminal acetylation
  BINDING 12: note = C-terminal amidation
AKRFFGYKRKFF

SEQ ID NO: 3 - protein, 12 aa, synthetic construct
  BINDING 1: note = N-terminal acetylation
  BINDING 12: note = C-terminal amidation
  SITE 12: note = X is beta-Naphthylalanine
  SITE 4: note = X is beta-Naphthylalanine
  SITE 5: note = X is beta-Naphthylalanine
AKRXXGYKRKFX

SEQ ID NO: 4 - protein, 12 aa, synthetic construct
  BINDING 1: note = N-terminal acetylation
  BINDING 12: note = C-terminal amidation
  SITE 4: note = X is beta-(4.4'-biphenyl)alanine
  SITE 5: note = X is beta-(4.4'-biphenyl)alanine
  SITE 12: note = X is beta-(4.4'-biphenyl)alanine
AKRXXGYKRKFX

SEQ ID NO: 5 - protein, 12 aa, synthetic construct
  BINDING 1: note = N-terminal acetylation
  BINDING 12: note = C-terminal amidation
  SITE 4: note = X is beta-diphenylalanine
  SITE 12: note = X is beta-diphenylalanine
  SITE 5: note = X is beta-diphenylalanine
AKRXXGYKRKFX